(19) United States
(12) Patent Application Publication
(10) Pub. No.: US 2018/0242874 A1
(43) Pub. Date: Aug. 30, 2018

(54) DEVICES, SYSTEMS AND METHODS FOR CORONARY AND/OR PULMONARY ABNORMALITY DETECTION UTILIZING ELECTROCARDIOGRAPHY

(71) Applicant: AUM Cardiovascular, Inc., Northfield, MN (US)

(72) Inventors: Marie Johnson, Northfield, MN (US); Jixing Yao, Northfield, MN (US); Nick Paulson, Minneapolis, MN (US); Mike Larson, Prior Lake, MN (US); Jordan Lippett, Maple Grove, MN (US)

(21) Appl. No.: 15/756,336

(22) PCT Filed: Aug. 26, 2016
(86) PCT No.: PCT/US16/48937

§ 371 Date: Feb. 28, 2018

Related U.S. Application Data
(60) Provisional application No. 62/211,545, filed on Aug. 28, 2015.

(51) Int. Cl. A61B 5/0432; A61B 5/00; A61B 7/04

(52) U.S. Cl. CPC A61B 5/0432; A61B 5/721; A61B 2560/0228; A61B 2560/0214; A61B 7/04

(57) ABSTRACT

Embodiments relate to non-invasive medical devices, systems and methods for the identification or detection of coronary artery disease, or other anatomical or physiological conditions. In an embodiment, a handheld coronary artery disease (CAD) detection device is used in a non-invasive manner to determine whether an internal coronary artery blockage or other condition is present. In an embodiment, a data transfer system comprises a handheld data collection device or other data collection device, and a networked or local computing system for storing and processing the recorded data. Supplemental devices, such as a tablet, can facilitate use, operation and data transfer between the data collection device and networked or local computing system.

### RELATED APPLICATION

The present application claims the benefit of U.S. Provisional Application No. 62/211,545 filed Aug. 28, 2015, which is hereby incorporated herein in its entirety by reference.

### TECHNICAL FIELD

Embodiments relate generally to detecting electric cardiovascular, valvular, peripheral, renal, carotid or pulmonary signals and a diverse cardiovascular characteristic to diagnose heart, lung, and other conditions. Embodiments relate more particularly to non-invasive or invasive medical devices, systems and methods for the detection of coronary artery and other diseases using both acoustic and electrical data.

### BACKGROUND

Cardiovascular disease is the leading cause of death in both men and women in the United States, and is a major cause of death throughout the world. According to a 2006 American Heart Association (AHA) report, approximately 80 million people in the United States have heart disease and in 2005, 864,480 people lost their lives. This accounts for 35.3 percent of all deaths or one of every 2.8 deaths in the United States according to the AHA. Cardiovascular cost the health care system approximately $368.4 billion in 2004, accounting for nearly a third of the trillion dollars spent on health care in the United States each year, again according to the AHA. Patient care accounts for 90% of this cost.

The health care system would benefit tremendously by identification of those individuals at high risk for coronary related attacks. Current evidence shows that established cardiac risk factors, such as certain abnormal levels of blood pressure, blood glucose and cholesterol and a history of smoking, possess a limited ability to estimate cardiac risk. In symptomatic patients with suspected cardiovascular disease, there are a variety of tests available to establish diagnosis. It remains a difficult problem, however, as clinical history and additional information is needed to establish the diagnosis, estimate prognosis and guide appropriate treatment. Coronary angiography is considered the “gold standard” for diagnosis, but it is invasive and costly and is an appropriate initial diagnostic study in only a minority of patients.

Other tests include exercise treadmill test, stress echocardiogram, computed tomography, calcium heart scanning and angiography. Each of these tests is ordered by clinicians after a patient is suspected to have Coronary Artery Disease (CAD). These tests vary in their accuracy with angiogram considered the gold-standard. Exercise electrocardiogram (ECG) testing is the most commonly used test because it is simple and inexpensive. This type of test is sometimes also referred to as EKG testing, and one of ordinary skill in the art will recognize that these terms are equivalent to one another. The patient must be able to exercise to at least 85 percent of the predicted maximal heart rate to rule out ischemic heart disease if the test is otherwise negative. For patients who cannot exercise, have baseline ECG abnormalities that could interfere with exercise ECG testing, or in whom the exercise ECG test suggests intermediate risk, a number of alternative noninvasive tests are available including echocardiography with exercise or pharmacologic, radionuclide myocardial perfusion imaging (rMPI), using either planar or photon emission computed tomographic as the imaging method, positron emission tomography (PET) or using coronary calcium scores. Many of these tests are also invasive, time-consuming and expensive, requiring trained personnel and capital equipment.

Moreover, the aforementioned hospital-centric devices are traditionally coupled to a hospital or caregiver network in order to facilitate data transfer and data flow of the recorded data. No built-in network for data transfer and data flow exists for non-hospital-centric devices. As a result, traditional portable devices often are required to contain both sensing and analyzing components, which can be difficult and costly to engineer. Further, field-ready devices often lack the computing power to adequately analyze the recorded data. In other cases, data transfer networks must be created ad-hoc. Therefore, there is a need for improved devices, systems and methods for the detection of coronary artery disease and other diseases.

### SUMMARY

Embodiments of the present application substantially address or meet the aforementioned needs of the industry. In an embodiment, a system comprises a handheld CAD detection device or other data collection device, and a networked system for storing, securely transferring, and/or processing the recorded data. Further, supplemental devices such as a hub, smartphone, dongle, or other mobile networked device can facilitate data transfer between the data collection device and network-based system.

In embodiments, a handheld CAD detection device can include both acoustic and electrocardiogram (ECG) sensors. By combining, co-considering and/or co-processing ECG and acoustic data, which can be measured concurrently at the same location, more accurate diagnoses of CAD and other conditions can be generated and qualitative results related thereto provided. In some embodiments, the CAD detection device can also include or be coupled with a camera, light sensor, temperature sensor, accelerometer, gyroscope, and/or various other sensors or devices as depicted herein. In embodiments, the aforementioned components can further assist in noise reduction or noise cancellation. In one particular embodiment, the CAD detection device can operate in conjunction with a tablet or other portable computing device communicatively coupled therewith, and noise reduction or noise cancellation also can be provided by features of the tablet or portable computing device.

The above summary is not intended to describe each illustrated embodiment or every implementation of each and every embodiment. The figures and the detailed description that follow more particularly exemplify these embodiments.

### BRIEF DESCRIPTION OF THE DRAWINGS

Embodiments may be more completely understood in consideration of the following detailed description of various embodiments in connection with the accompanying drawings, in which:

FIG. 1 is a block diagram of a system for the detection of coronary artery disease according to an embodiment.

FIGS. 2A and 2B are perspective views of a CAD detection device and docking station according to an embodiment.

FIGS. 3A and 3B are perspective views of a CAD detection device according to an embodiment.

FIG. 4 is a perspective view of a CAD detection device according to an embodiment.

FIG. 5 is an exploded view of a CAD detection device according to an embodiment.

FIGS. 6A-6D depict ECG sensor arrangements of a CAD detection device according to three embodiments.

FIG. 7 is a block diagram of a CAD detection device according to an embodiment.

FIG. 8A is a perspective view of a docking station according to an embodiment.

FIGS. 8B and 8C are top views of a CAD detection device and a docking station according to an embodiment.

FIG. 8D is a top view of a CAD detection device according to an embodiment.

FIGS. 9A and 9B depict an instruction booklet with built in wireless signal tags according to an embodiment.

FIG. 9C depicts a system comprising tablet computing device, a CAD detection device, and a networked or local computer system according to an embodiment.

FIG. 9D depicts an example of a user view when using an augmented reality system with a CAD detection device according to an embodiment.

FIG. 10 is a graphical representation of a normal ECG profile produced by an embodiment of a CAD detection device.

FIG. 11 is a graphical representation of a diseased ECG profile produced by an embodiment of a CAD detection device.

FIG. 12 is an information flow chart corresponding to a CAD detection device according to an embodiment.

While embodiments are amenable to various modifications and alternative forms, specifics thereof have been shown by way of example in the drawings and will be described in detail. It should be understood, however, that the intention is not to be limited to the particular embodiments described. On the contrary, the intention is to cover all modifications, equivalents, and alternatives falling within the spirit and scope of the appended claims.

### DETAILED DESCRIPTION OF THE DRAWINGS

Embodiments relate to non-invasive and invasive medical devices, systems and methods for the detection of coronary artery and other diseases in both humans and in veterinary applications. In an embodiment, a handheld coronary artery disease (CAD) detection device is used in a non-invasive manner to determine and provide a qualitative result with respect to whether an internal coronary artery blockage is present. In another embodiment, the CAD device can be used to diagnose valvular, peripheral, carotid, renal or pulmonary disease. In an embodiment, a data transfer system comprises a handheld CAD detection device or other data collection device, and a networked system for storing and processing the recorded data. The handheld CAD detection device can measure both acoustic and electrocardiogram (ECG) signals, and these signals can be used to identify at least one pattern based on both the ECG and acoustic signals to assess physiology and anatomy changes associated with CAD or other disorders that can be sensed by obtaining electrical and acoustic data from the test subject, accurately and efficiently as compared to conventional techniques.

In alternative embodiments, various other anatomical and/or physiological conditions can be observed and diagnosed, or data related thereto can be presented for physician or other medical professional review and analysis. For example, instead of CAD, various other cardiac, pulmonary, vascular, or other conditions can be detected by a combination of sensors similar to those described below. The combination of multiple modes measured simultaneously can be more informative that a single type of information, or even multiple modes measured at separate times. As such, with respect to the embodiments depicted below, the electrical signal mode from ECG sensors combined with acoustic data from the acoustic sensors measured at the same time enhances the ability of the overall system to diagnose CAD, whereas in alternative embodiments acoustic, electrical, temperature, pressure, or any of a number of other modes could be measured concurrently in order to better diagnose other anatomical or physiological conditions.

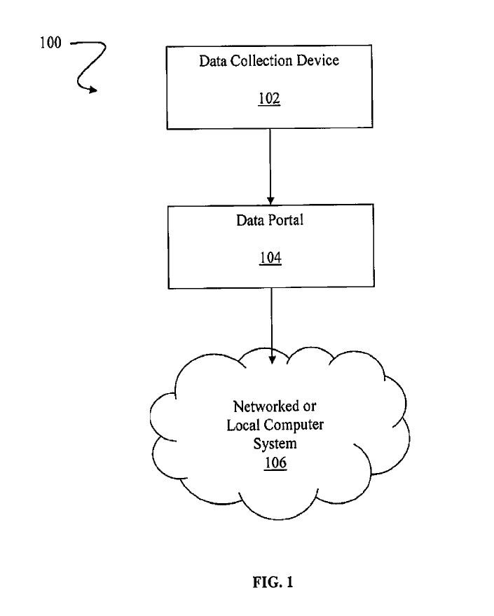

A block diagram of a data transfer system 100 is depicted in FIG. 1, according to an embodiment. Data transfer system 100 generally comprises a data collection device 102, a data portal 104, and a networked or local computer system 106 which could be, for example, a cloud.

Data collection device 102 comprises a device for recording, sensing, or otherwise collecting data. In an embodiment, data collection device 102 is configured to collect data from a patient. In embodiments, the patient or test subject can be human or another animal. In an embodiment, data collection device 102 comprises a CAD detection device, as will be described further below. Data collection device 102 can be configured to be wireless and portable so as to collect data in remote places or places without a traditional hospital infrastructure.

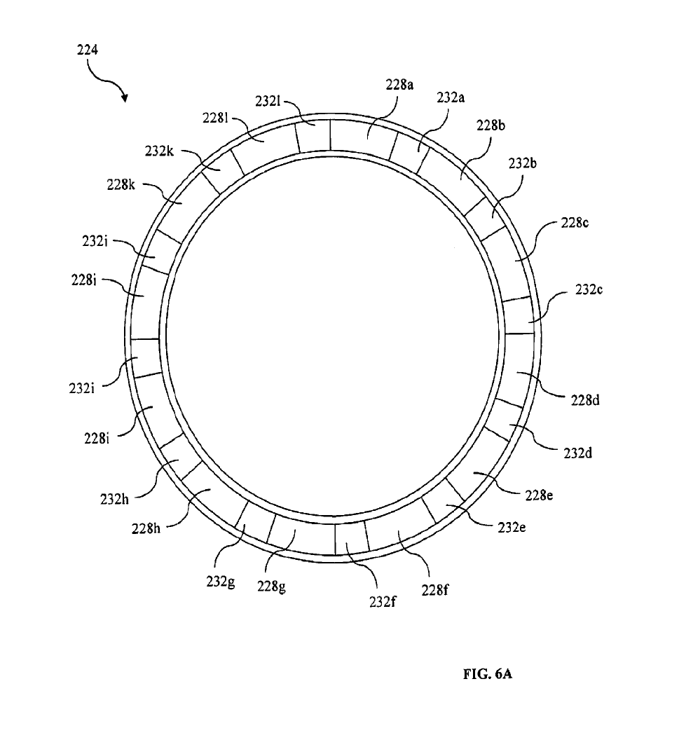

Data portal 104 comprises a base station or portal configured to interface to at least one data collection device 102. In embodiments, data portal 104 comprises a desktop computer, data hub, laptop computer, smartphone, personal digital assistant (PDA), tablet, watch, wearable electronic device, or other suitable device. In embodiments, data portal 104 acts as a pass-through to transfer collected data from one or more data collection devices 102 to networked or local computer system 106. In other embodiments, data portal can consolidate, combine, and/or package data, as will be described in more detail with respect to FIGS. 5E and 5F, before the data is transmitted.

In one embodiment of a pass-through device, data portal 104 can be a dedicated pass-through device. Dedicated pass-through devices can be left in standby or always-on mode, and serve only to transmit information. Dedicated pass-through devices are distinct, then, from multi-use devices such as smartphones, smartwatches, or other wearable data transmission devices, which are used in alternative embodiments. Plug-in or battery-operated devices can be positioned in a location proximate to expected testing operations. For example, a commercially available hub device can be plugged into a wall outlet in an examination room where data collection device 102 is to be used. Such pass-through devices can be configured to activate upon wirelessly receiving data from associated devices (such as data collection device 102) and pass on the information to a remote server (such as networked or local computer system 106). In embodiments, data transfer can be accomplished via WiFi networks, cellular networks, mobile data networks, wired Ethernet or telephone line networks, or any other transmission medium to networked or local computer system 106.

In embodiments, data collection device 102 and data portal 104 are operably coupled by a communication network and suitable hardware. For example, both data collection device 102 and data portal 104 can comprise Universal Serial Bus (USB), Firewire, Bluetooth, serial, EEPROM, WI-FI, or any other appropriate hardware, software, and suitable interfaces. In embodiments, data collection device 102 is configured with minimal data transmission hardware (i.e., with hardware necessary to transmit to data portal 104), so that data collection device 102 can be more inexpensively and efficiently produced. In such embodiments, data portal 104 can be configured with additional communication hardware to receive data from data collection device 102 and transmit the data to any number of configurations of networked or local computer system 106. In other embodiments, data collection device 102 can include some data storage capability, enabling the storage of a desired number of previous test results, software, or other information.

Networked or local computer system 106 can also include data storage and/or a processing engine to analyze the data provided by data collection device 102 remotely. In embodiments, networked or local computer system 106 is configured to store and process data received from data collection device 102 via data portal 104, and in some embodiments networked or local computer system 106 is configured to store, process and/or aggregate data received from a plurality of data collection devices 102 via one or more data portals 104. In embodiments, networked or local computer system 106 is configured to produce a report, analysis, diagnosis, or other output based on the received data. In embodiments, networked or local computer system 106 therefore comprises an analytics engine. In alternative embodiments, networked or local computer system 106 could be any other networked system, such as a wired system, a remote server, or even a local computer drive, rather than a system that is accessed wirelessly.

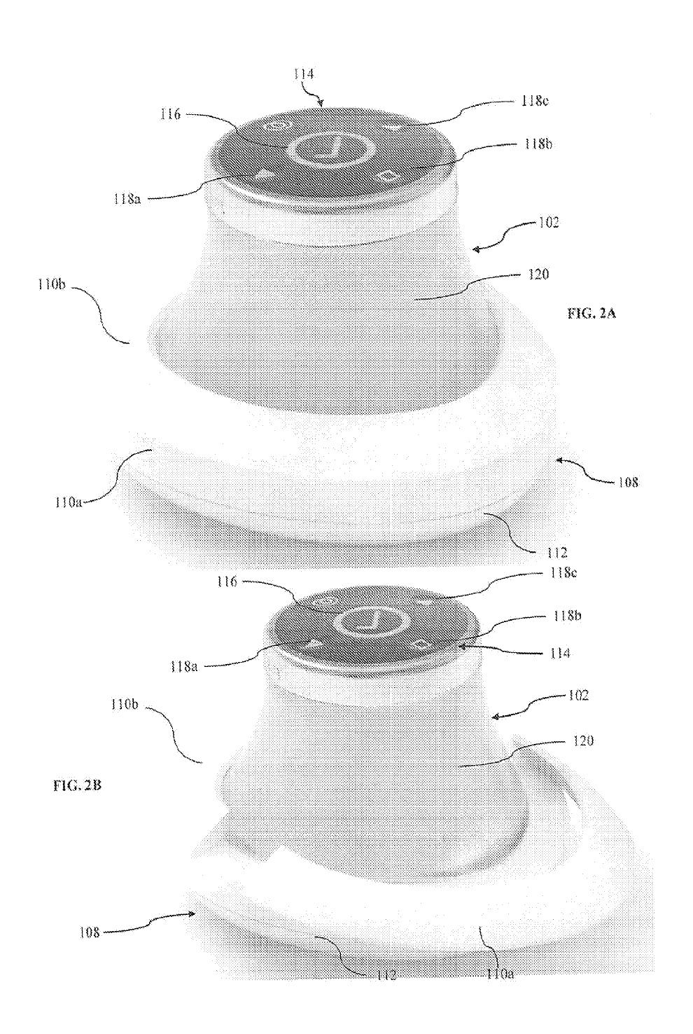

FIGS. 2A and 2B depict one embodiment of data collection device 102. Data collection device 102 is shown coupled to docking station 108, which includes a first retaining member 110a, a second retaining member 110b, and a base 112. In particular, FIGS. 2A and 2B illustrate face 114, which includes display 116 and inputs 118a-118c. As shown in FIG. 2, data collection device 102 also includes sidewall 120.

Docking station 108 can optionally be used for one or more of a variety of functions in a CAD detection device. For example, docking station 108 can be used to recharge data collection device 102 in embodiments. Alternatively or additionally, docking station 108 can be used to perform diagnostic tests of data collection device 102 in embodiments. Furthermore, docking station 108 can provide a stable, dry, quiet environment in which data collection device 102 can be housed when not in use, in embodiments.

Docking station 108 as shown in FIGS. 2A and 2B can be removably, selectively secured to data collection device 102 using first retaining member 110a and second retaining member 110b. As shown in FIG. 2A, when data collection device 102 is not being used to collect data from a patient, first retaining member 110a and second retaining member 110b are operatively connected to one another to form a closed ring around sidewall 120 of data collection device 102 and restrict movement of data collection device 102 relative to base 112.

To remove data collection device 102 from docking station 108, at least first retaining member 110a can be detached from base 112. Retaining members 110a and 110b are one of a number of embodiments that can be used to couple data collection device 102 to base 112 for recharging, diagnostic testing, and/or data transfer. In various embodiments, there can be relatively more or fewer retaining members, or even no retaining members, and the retaining members can have alternative configurations, shapes, or sizes than those depicted. In some embodiments, one or more of the retaining members can remain in a fixed position while others are movable or removable (e.g., first retaining member 110a can be movable while second retaining member 110b can be affixed to base 112). In embodiments, retaining members 110a and/or 110b can be attached to base 112 via a hinge or a track system, for example, allowing relative movement between base 112 and the retaining member(s) 110a and/or 110b without complete detachment of retaining members 110a or 110b from base 112.

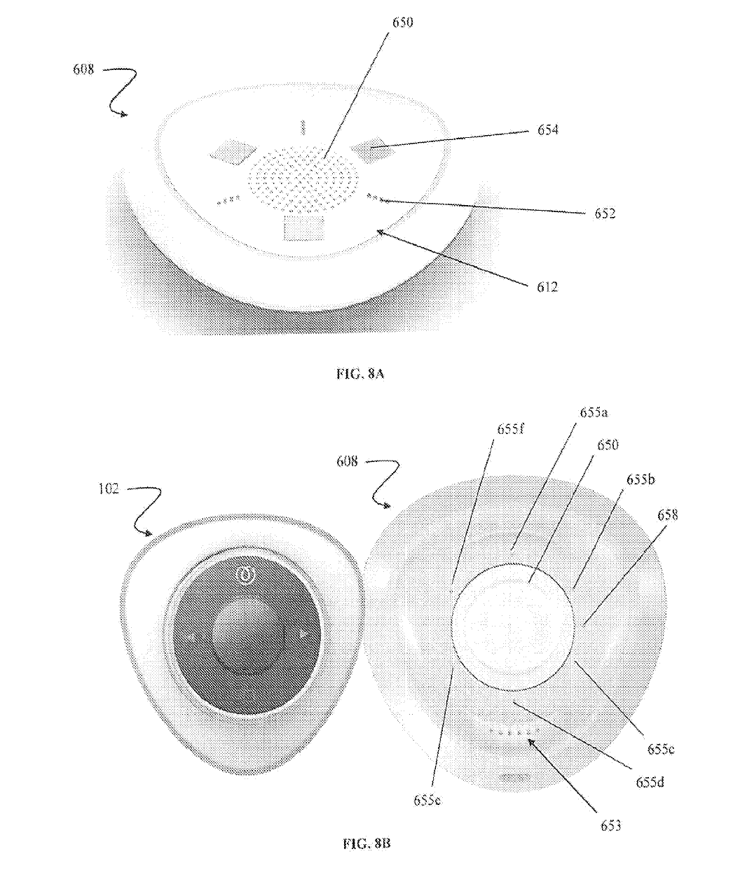

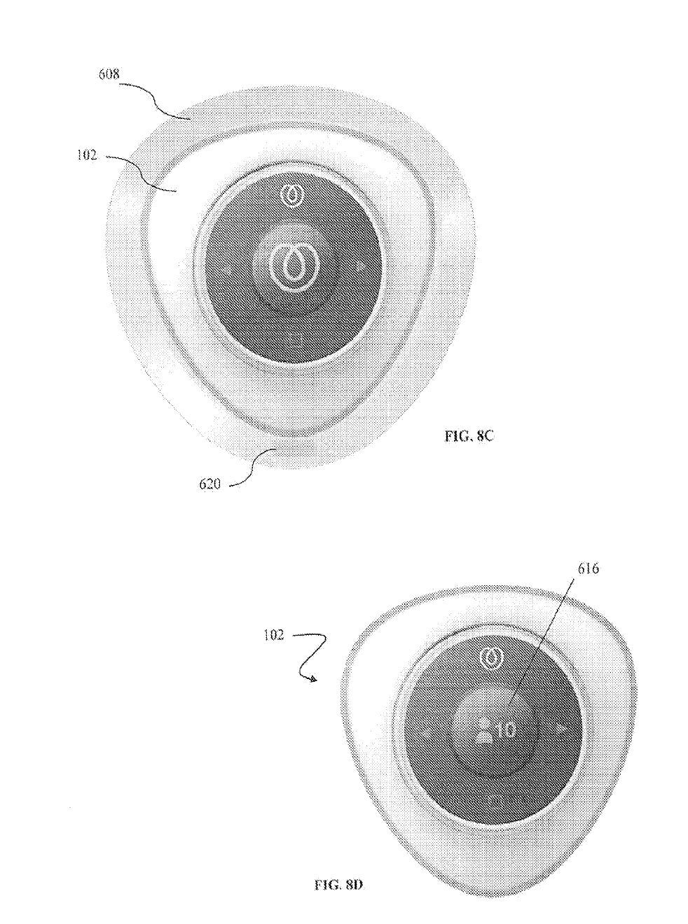

In embodiments, data collection device 102 includes one or more sensors that can be tuned for low signal levels (such as electrical or acoustic signals from a heart) compared to ambient electromagnetic and acoustic signals. As such, docking station 108 can provide an electromagnetically- and/or acoustically-dampened environment such that when testing is not actively taking place, the sensors are sheltered from potentially damaging signals or substances. Furthermore, as will be described in more detail with respect to FIG. 8, docking station 108 can carry out testing and calibration of data collection device 102, or charging of data collection device 102, in embodiments. In embodiments, docking station 108 can include data processing, storage, and/or transmission hardware such that docking station 108 incorporates all of the functions of data portal 104. In these embodiments, a separate data portal 104 may not be necessary in carrying out CAD or pulmonary disease detection.

Data collection device 102 of FIGS. 2A and 2B includes a face 114 that can be used as a graphical interface as well as input and/or output of information. Face 114 includes a variety of other components, such as display 116 and inputs 118a-118c, in the embodiments shown in FIGS. 2A-2B.

In embodiments, an intuitive and simple graphical user interface on or communicatively coupled to a handheld CAD detection device provides an interface for controlling and operating the device. Display 116 can output graphical indications of test status, charging status, or other information relating to data transmission, calibration, or data quality, among others. In embodiments, display 116 can display in multiple colors (e.g., blue for a “ready” status, red to indicate an error or that data collection device 102 needs to be recharged). In embodiments, display 116 can comprise a different size or shape. In some embodiments, display 116 facilitates data input as well as display. For example, display 116 or parts thereof can be touch-sensitive.

Inputs 118a-118c provide another mechanism for interacting with and entering information and commands into data detection device 102. A practitioner using data collection device 102 can use inputs 118a-118c to indicate that data collection should begin, or that data is ready for transmission or analysis, for example. In one embodiment, a practitioner or user can turn device 102 on or off via input 118b; and begin, restart or navigate within data collection via forward and back inputs 118a and 118c, respectively. In other embodiments, different or additional information or commands can be provided to device 102 via inputs 118a-118c. At the same time, display 116 can provide visual feedback, prompts and/or directions to the user of the commands received, next user step to be taken, or other information related to device status or operation. In alternative embodiments, there could be relatively more or fewer inputs 118.

Sidewall 120 is a part of data collection device 102 in the embodiment shown in FIGS. 2A and 2B. Sidewall 120 can be shaped to engage with docking station 108, including first and second retaining members 110a and 110b. Sidewall 120 also provides a location for a medical practitioner or other user to hold data collection device 102 during testing. In embodiments, sidewall 120 can be made of a variety of electrically insulating materials, such as silicone rubber or a thermoset or thermoplastic polymer with a silicone rubber overmold. In embodiments, sidewall 120 is ribbed or textured or partially hollow to increase internal volume for internal components. In some embodiments, sidewall 120 can comprise, or be coated partially or fully with, a material that is grippable or enhances grippability by a user, to make it less likely for the user to drop device 102 during use or handling. For example, sidewall 120 can comprise Versaflex in one embodiment. A similar material can be provided on an underside of a docking station 108 (discussed in more detail below) that can be used with device 102, in order to prevent docking station 108 from sliding or slipping when placed or resting on a surface. In general, the materials of sidewall 120 and device 102 as a whole are biocompatible.

The embodiment of data collection device 102 as shown in FIGS. 2A-2B is physically compact in comparison to conventional cardiography or angiography equipment, and can be recharged and protected by docking station 108 when not in use. As such, data collection device 102 is a relatively robust and convenient alternative to the more expensive, bulkier, or less reliable alternatives that are presently available.

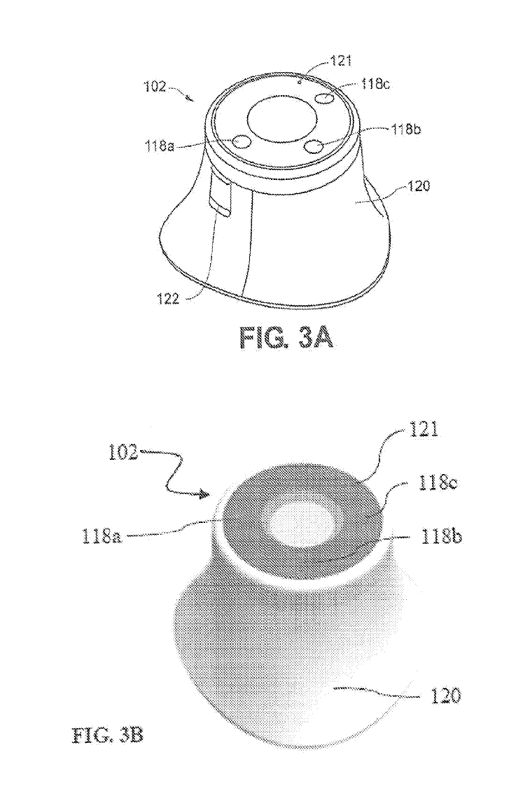

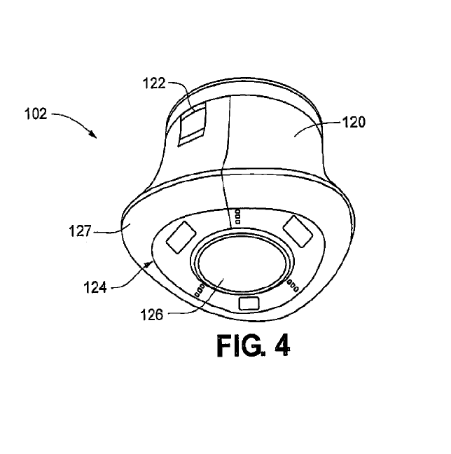

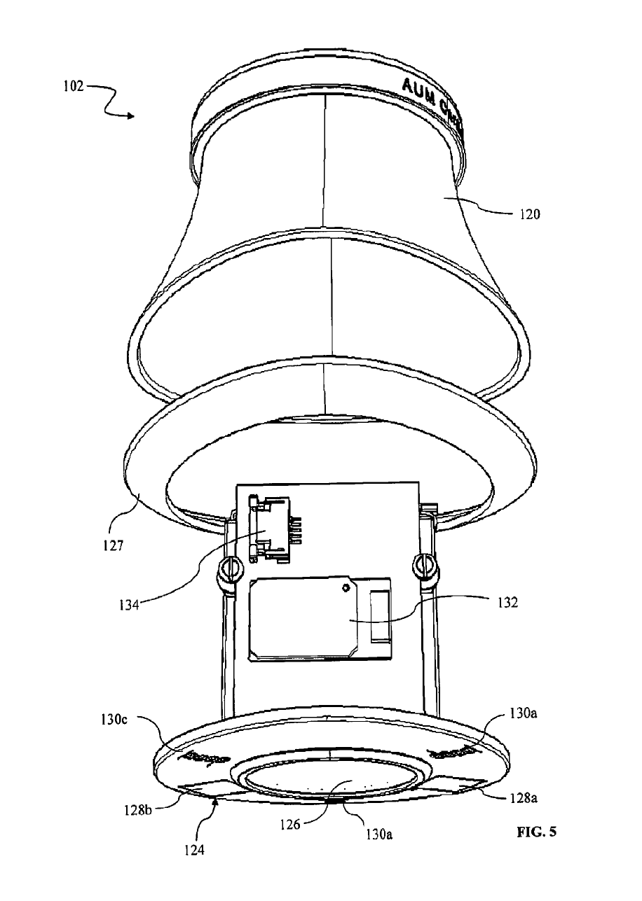

FIGS. 3A-4 are additional views of data collection device 102, including the features previously shown with respect to FIGS. 2A-2B. Additionally, FIGS. 3A-4 depict a secondary acoustic sensor, microphone 121, as well as port 122, ECG sensor panel 124, acoustic sensor panel 126, and flange 127.

Microphone 121 can be used to compensate for ambient noise and vibrations incident upon data collection device 102 during testing. In embodiments, microphone 121 can gather information about the ambient acoustic environment and use such data to correct input from other acoustic sensors (such as those associated with acoustic sensor panel 126, described below). Microphone 121 can also be used during calibration procedures when data collection device 102 is positioned in docking station 108, as previously described with respect to FIGS. 2A and 2B. In embodiments, where an ambient noise level is too high for accurate testing, microphone 121 can send a signal causing display 116 to indicate that testing cannot commence or should be retried in a quieter location. In embodiments, sound-proofing materials can be used in sidewall 120 and/or inside retaining members 110a and 110b to create an acoustic seal, which can block ambient noise or at least reduce the ambient noise level to increase the accuracy of an acoustic test.

Port 122, as shown in FIGS. 3 and 4, is a USB port. In some embodiments, data can be transferred either to or from data collection device 102 via a wired connection such as USB, or an Ethernet cable, a telephone cable, or some other wired connection, and in some embodiments a USB port can be used for charging a battery of the device 102. In embodiments, data collection device 102 need not have any port 122 whatsoever, and can be preloaded with all necessary software and firmware to perform data transfer exclusively wirelessly.

An ECG sensor panel 124 and an acoustic sensor panel 126 can be arranged on the same surface of data collection device 102 radially inward of flange 127, and are configured to collect electrical and acoustic information from a patient, respectively. ECG sensor panel 124 includes at least one electronic sensor, such as an electrical potential sensor or a Plessey sensor, which can measure electrical activity related to heart function, for example. Acoustic sensor panel 126 includes or covers an acoustic sensor or sensors configured to measure acoustic activity related to heart function, for example. ECG sensor panel 124 can include any of a variety of ECG sensors, including RED DOT electrodes, for example, among other electrodes and/or sensors that will be known to those of skill in the art.

Data from ECG sensor panel 124 and acoustic sensor panel 126 can be combined or used together to generate a more accurate and/or informative diagnosis of heart function and potential disorders than either type of data in isolation. ECG data can be used to determine the anatomical location of a blockage detected by acoustic sensing, for example. ECG sensor panel 124 can measure electronic signals indicative of certain heart conditions such as arrhythmia, ischemia, or infarction, for example, whereas acoustic sensor panel 126 can detect acoustic evidence such as turbulence, leakage, or arrhythmia that are indicative of other heart conditions. Combining, co-processing, and/or co-considering these two data, such as where they are acquired concurrently and at or near the same location(s), can provide a more accurate diagnosis than either type of test in isolation. Accordingly, the acoustic sensor panel 126 is arranged adjacent the ECG sensor panel 124 in embodiments, wherein “adjacent” is defined for purposes of this application as near enough to one another so as to acquire data that correspond to the same test site on the same patient concurrently. For example, in the arrangement shown in FIG. 4, a single face of data collection device 102 at the base of sidewall 120 includes both ECG sensor panel 124 and acoustic sensor panel 126 that can collect data from a patient contemporaneously.

Data from ECG acoustic sensor panel 126 can be compared to the ambient sound measured at microphone 121. Microphone 121 can provide an indication whether a signal at acoustic sensor panel 126 is a result of ambient conditions or whether it is from the patient being tested.

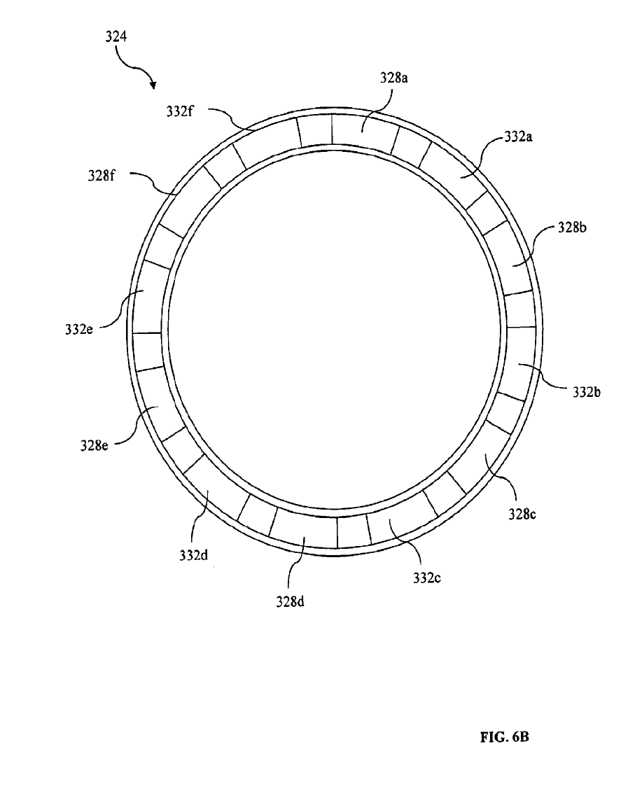

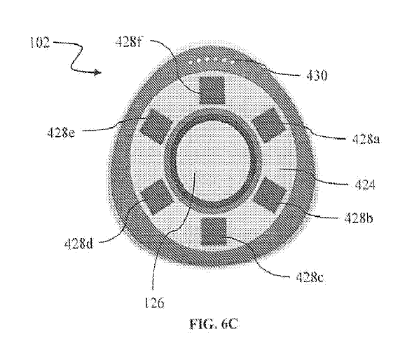

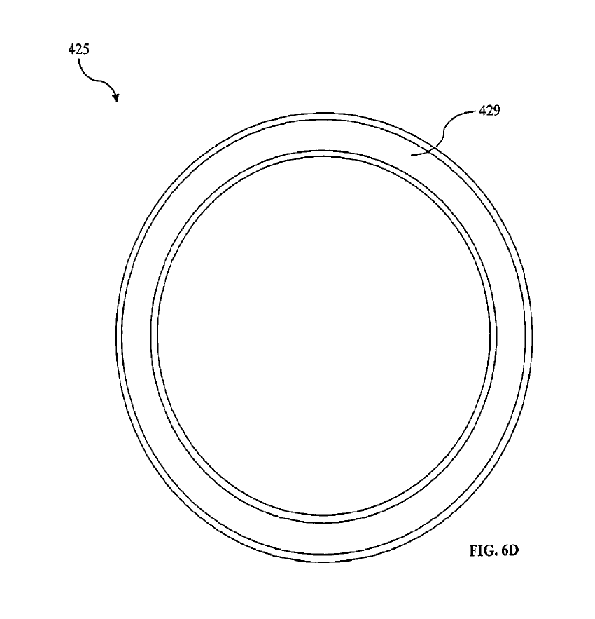

FIG. 5 is an exploded view of data collection device 102. Individual sensors 128a and 128b and pin sensors 130a-130c are shown on ECG sensor panel 124. Plessey sensors 128a and 128b can collect data related to electromagnetic fields. Pin sensors 130a-130c (also referred to collectively as pin sensors 130) can also collect electrical potential information or, in some embodiments, pin sensors 130a-130c can be connected to corresponding components in base 112 of docking station 108 (FIGS. 2, 8A-C) to charge a battery in data collection device 102 or to upload data from data collection device 102 to base 112 or download data from base 112 to data collection device 102. In embodiments, pin sensors 130a-130c can be used to send or receive commands to and from docking station 108. In one embodiment, pin sensors 130 on device 102 comprise female pin sensor portions that correspond with male pin sensor portions on docking station 108 (see the embodiments of FIGS. 8A and 8B), though this male/female relationship can be reversed, or another type of sensor can be used, in other embodiments. The number and arrangement of sensors in ECG sensor panel 124 can vary in embodiments. FIGS. 6A-6C, for example, depict three alternative ECG sensor arrangements, and FIG. 6C also depicts an alternative arrangement of pin sensors 430.

FIG. 5 also shows memory 132 and wireless chip 134. In use, acoustic sensor panel 126, Plessey sensors 128a and 128b, and pin sensors 130a-130c can each send measured data to memory 132 and/or wireless chip 134. In addition to memory 132 and wireless chip 134, data collection device 102 can also house a battery, circuit boards, a processor, or other components that are used to acquire, store, process, or transmit collected data.

FIG. 6A depicts an ECG sensor panel 224 that includes twelve ECG sensors 228a-228l and twelve ground contacts 232a-232l. In a typical ECG test, having twelve ECG sensors 228a-228l is standard. An arrangement of twelve ECG sensors 228a-228l can provide the information needed to diagnose electrical irregularities in the heart of a subject by comparing the signal at one of the twelve ECG sensors 228a-228l with another of the twelve ECG sensors 228a-228l. Each such comparison is referred to as a “channel,” and specific signals on specific channels can be indicative of specific cardiac disorders.

In conventional ECG tests, the ECG sensors may be arranged further from one another, and ground can be at a remote location such as a wire held in the hand of the test subject or a grounded pad or other device on which the test subject sits or lays. Data collection device 102, in contrast, incorporates the electrical ground into the comparatively small footprint of ECG sensor panel 224. In order to prevent the electrical ground value from varying due to the electrical signal from the patient, and to prevent the signal from one of the ECG sensors 228a-228l from affecting the signal at an adjacent ECG sensor 228a-228l, twelve ground contacts 232a-232l are arranged in an interdigitated fashion between the ECG sensors 228a-228l. In embodiments, all twelve ground contacts 232a-232l are electrically connected in a “broken ring” or other arrangement in which the electrical connections between the ground contacts 232a-232l do not form a closed loop that could act as an antenna.

FIG. 6B is an alternative embodiment of an ECG sensor panel 324 having six ECG sensors 328a-328f and six ground contacts 332a-332f. As previously described with respect to the embodiment shown in FIG. 6A, the ECG sensors 328a-328f are interdigitated with the ground contacts 332a-332f, preventing the ground reference from varying with heart function and also electrically isolating each of the ECG sensors 328a-328f from the others. ECG sensor panel 324 facilitates six-lead ECG measurements. Six-lead ECG is an alternative to 12-lead ECG which requires fewer sensors to perform.

FIG. 6C is an alternative embodiment of an ECG sensor panel 424 having six ECG sensors 428a-428f. In the embodiment of FIG. 6C, ECG sensor panel 424 can comprise or be coupled with a single ground plane, within which ECG sensors 428a-428f are isolated or separated from one another. For example, a ground plane can be arranged within device 102 under ECG sensor panel 424, with ECG sensor panel 424 coupled with the ground plane to ground ECG sensor panel 424 itself.

FIG. 6D is an alternative embodiment of an ECG sensor panel 425 having a single face 429. Face 429 can be more aesthetically pleasing and also facilitates measuring electrical potential or electromagnetic field at any point on face 429. Face 429 can cover stationary or movable contacts housed within a data collection device (e.g., data collection device 102) that can measure these data at any of a variety of positions and times.

In embodiments, face 429 has a resistivity that is tuned to prevent electrical grounding between the various contacts, while permitting measurement through face 429 of the electrical potential at a variety of locations, or grounding to the test subject. This can be accomplished either by making face 429 of a material having a relatively high resistivity, and/or separating the various ECG and ground contacts behind face 429 sufficiently far from one another, for example. In alternative embodiments, face 429 can be made of an anisotropic material, facilitating current flow through face 429 but not along face 429 between electrical contact points. In still further embodiments, face 429 can appear continuous while containing regions of high and low resistivity, to facilitate current flow through face 429 but not throughout face 429 between electrical contact points.

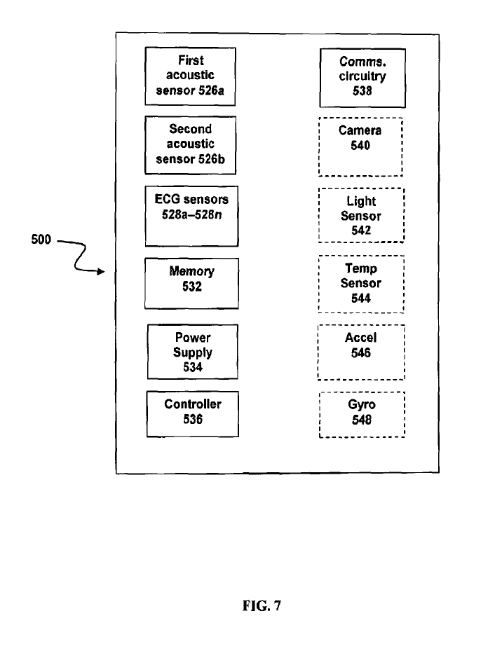

Referring to FIG. 7, a CAD detection device 500 comprises, in one embodiment, a first acoustic sensor 526a, a second acoustic sensor 526b, a set of ECG sensors 528a-528n, a memory 532, a power supply 534, a controller 536, communications circuitry 538, a camera 540, a light sensor 542, a temperature sensor 544, an accelerometer 546, and a gyroscope 548. CAD detection device 500 can comprise more or fewer components in various embodiments. For example, one or more of camera 540, light sensor 542, temperature sensor 544, accelerometer 546, and or gyroscope 548 can be omitted in embodiments.

In the embodiment depicted in FIG. 7, CAD detection device 500 comprises two acoustic sensors 526a and 526b. One pressure sensor 526a collects data related to the presence or absence of a turbulent pressure wave in a coronary artery. Blood flow is periodic in time and laminar in flow. A blockage can act as a nozzle within an artery, causing turbulence to occur. This turbulence creates a turbulent pressure wave that can be detected by acoustic sensor 526a. Acoustic sensor 526b, in one embodiment, is included for noise cancelling, such as active and/or passive noise cancelling to filter out ambient noise and other irrelevant information.

In other embodiments, one or both of acoustic sensors 526a and 526b can comprise acoustic or other sensors suitable for data collection, active and/or passive noise cancellation and/or other tasks related to the operation of CAD detection device 500. For example, in one embodiment of noise cancellation, background noise is sampled by at least one sensor 526a or 526b and subtracted from an overall signal in order to cancel noise and improve signal quality. In this and other embodiments, one or both of sensors 526a and 526b and/or related components and circuitry are mechanically potted, or encapsulated, to improve noise cancellation. In embodiments, one or both of sensors 526a and 526b are electrically shielded to reduce noise. Still other sensors in addition to sensors 526a and 526b also can be included in device 500, even though they are not specifically depicted in FIG. 7.

Automatic signal strength detection circuitry and signal processing techniques can also be implemented in embodiments to improve signal quality. For example, in one embodiment, a signal kernel or shape is determined from signal samples and compared with an overall signal or pattern to determine whether the sample fits. If not, new data can be collected, existing data can be otherwise processed, and/or detection device 500 can be repositioned, among other tasks.

Therefore, active noise cancellation can be implemented in embodiments of CAD detection device 500 as described above and elsewhere herein. Passive noise cancellation can be implemented in combination with active noise cancellation, or on its own, in other embodiments. Materials such as, but not limited to, rubber, foam, sponge, glass fiber, ceramic fiber, mineral fiber, vinyl, tape, or sound-absorbing coatings and pastes or combinations thereof can be disposed proximate one or both of acoustic sensors 526a and 526b or otherwise suitably arranged on or within device 500 in order to passively reduce noise.

In embodiments, one or both of acoustic sensors 526a and 526b or another sensor of device 500 can comprise pressure sensors or other sensors suitable for sensing the pressure applied by a user to CAD detection device 500 in preparation for or during patient scanning. In embodiments, one or both of sensors 526a and 526b can comprise pressure sensors or other sensors suitable for sensing the pressure applied by a user to detection device 500 in combination with the ability to collect data related to the presence or absence of a turbulent pressure wave in a coronary artery. In other embodiments, an additional sensor or plurality of sensors is configured for sensing the pressure applied by a user to detection device 500 in preparation for or during patient scanning.

In embodiments, the pressure applied by a user is measured and compared against a minimum value representative of a typical amount of minimum pressure to establish a signal, and a maximum value representative of a typical amount of maximum pressure so as to not max out the measured signal. If the measured pressure exceeds the maximum value, an error message can be displayed to the user via a user interface. Likewise, if the measured pressure is below the minimum value, an error message can be displayed to the user via the user interface. Other threshold minimum and maximum values can also be used, depending on the patient, user, or other appropriate factors.

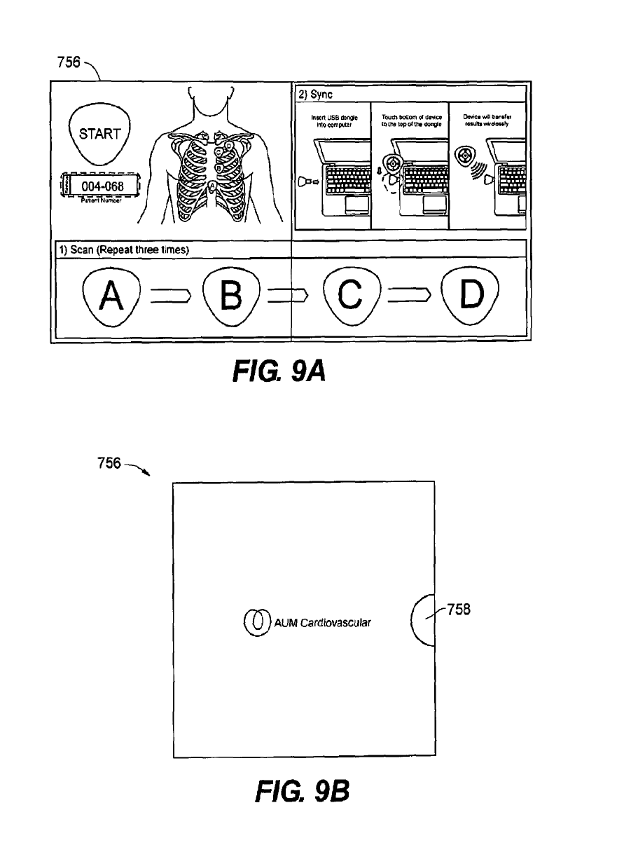

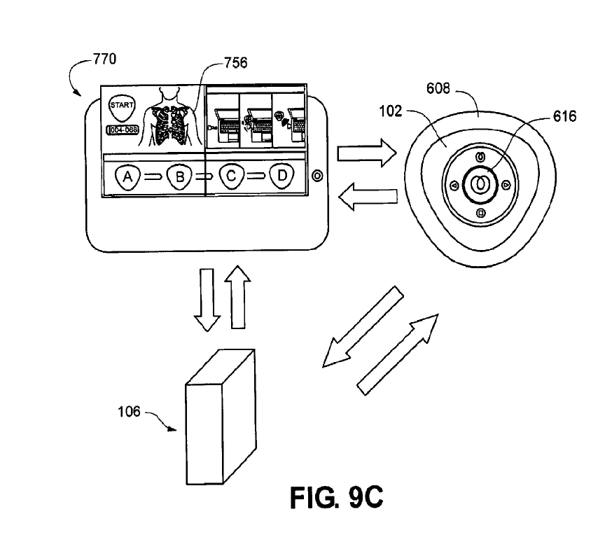

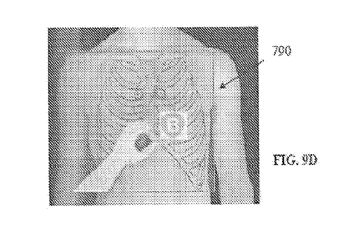

ECG sensors 528a-528n can provide data relating to electrical activity in the heart. In one embodiment, there can be six ECG sensors 528a-528n. The electrical activity of the heart or other anatomical feature of a human or animal can be modeled by analyzing the electrical outputs of each of the ECG sensors 528a-528n at each of the positions identified in a sequence guide (see, e.g., FIGS. 9A and 9B), in embodiments.

Memory 532, as previously described with respect to the memory (132) of FIG. 5, can store information from one or more tests. In addition, memory 532 can store test method information or software to interface with a wireless network or connected devices, for example.

Power supply 534 can comprise a battery in embodiments, such as a rechargeable battery or a replaceable battery. Rechargeable power supply 534 can be inductance-style, two-pin, charge by computer, and/or charge by AC wall outlet, or some other suitable charging configuration. In embodiments, power supply 534 can be powered or recharged through inductive charging. Power supply 534 can also be configured to allow for multiple different charging schemes. For example, CAD detection device 500 can interface with a charging station by physical coupling or cable, and/or CAD detection device 500 can couple by USB or other cable to a computer, docking station, wall outlet or other source of power. In embodiments, one power supply 534 is electrically shielded to reduce noise.

Controller 536 controls the operation of CAD detection device 500. During scanning, controller 536 can control a graphical user interface (GUI), a timer and the general operation of CAD detection device 500. In embodiments, the GUI is electrically shielded to reduce noise. Controller 536, via the GUI and/or an audible indicator, can also prompt a user to carry out various tasks, such as to apply CAD detection device 500 to one or more of patient scan areas in a sequence, to move on to a next guide area, tag or patient area, to rescan a particular guide area, tag or patient area, to reposition CAD detection device 500 if data of sufficient quality is not detected, to recharge CAD detection device 500 and other functions. CAD detection device 500 further comprises a timer, such as part of controller 536, for each scan to ensure that sufficient data is collected at each scan site. In one embodiment, this timer can automatically start as soon as data of a sufficient quality is detected by first acoustic sensor 526a, though other procedures can be used in other embodiments.

Controller 536 can be subservient to and/or operate in conjunction with an external controller coupled by wire or wirelessly in embodiments. In embodiments, controller 536 can carry out processing of collected data, for example to determine a presence of coronary artery disease based on data sampled by at least one of sensors 526a and 526b at the patient data acquisition locations associated with an identification areas guide (see, e.g., FIG. 9), though in other embodiments data processing is carried out external to CAD detection device 500, as described above. Controller 536 operates in cooperation with memory 532, which stores collected data until it is transferred to an external device or manually or automatically deleted and can be of any suitable type. Controller 610 also can correlate data sampled by first and second acoustic sensors 526a and/or 526b with a data acquisition location using the identification element, such as a sequence guide (see, e.g., FIGS. 9A and 9B).

Communication circuitry 538 is configured to transfer data to and from CAD detection device 500. For example, after scan data is collected for a particular patient and stored in memory 532, the raw scan data can be packaged and transferred wired or wirelessly to, for example, data portal 104 or networked or local computer system 106 for processing and determination of whether CAD may be present.

In another embodiment, CAD detection device 500 optionally further comprises a camera 540, photo or optical sensor, or other similar device. In embodiments, camera 540 can be configured to read or detect barcodes, such as one or more QR codes on a sequence guide (see, e.g., FIG. 9). In an embodiment, instead of visually moving through the stages of such a sequence guide as part of the sequence scanning, a QR code can be scanned by camera 616 and the subsequent sensing of the appropriate portion of the patient then can be conducted in any order. In embodiments, camera 540 is further configured for detecting or aligning to a portion of the patient's body. In embodiments, camera 540, along with appropriate software executable by controller 536, is configured to detect markers, flags, or any other suitable landmark of the patient to aid in aligning CAD detection device 500 to the patient. In another embodiment, camera 540 is configured to detect position of motion relative to the patient.

In another embodiment, CAD detection device 500 optionally further comprises a light sensor 542. In embodiments, light sensor 542 can be utilized to detect the proximity of CAD detection device 500 to the body of the patient or to a docking station (e.g., docking station 608 previously described with respect to FIG. 8). For example, a greater amount of light will be detectable by light sensor 542 when CAD detection device 500 is further from the patient. Likewise, less light will be detectable by light sensor 542 when CAD detection device 500 is proximate the patient. In embodiments, light sensor 542 can be a photoplethysmographic sensor. Such light sensing can be utilized to properly place and position CAD detection device 500.

In another embodiment, CAD detection device 500 optionally further comprises a temperature sensor 544. In embodiments, temperature sensor 544 can be utilized to detect the proximity of CAD detection device 500 to the body of the patient. For example, a lower temperature will be detectable by temperature sensor 544 when CAD detection device 500 is further from the patient. Likewise, a higher temperature will be detectable by temperature sensor 544 when CAD detection device 500 is proximate the patient. Such temperature sensing can be utilized to properly place and position CAD detection device 500.

In another embodiment, CAD detection device 500 optionally further comprises one or more accelerometers 546. In embodiments, accelerometer 546 is configured to detect acceleration of CAD detection device 500. For example, if accelerometer 546 detects an acceleration when CAD detection device 500 is in a data sense mode, it can be determined that CAD detection device 500 has moved or shifted during sensing and the data measured during that point may have errors or otherwise be incorrect. Such acceleration sensing can be utilized to properly place and position CAD detection device 500. In embodiments, an indication of a shift can be provided to the user via display 116 of CAD detection device 500. In embodiments, accelerometers 546 can also be used for acoustic detection.

In an embodiment, CAD detection device 500 optionally further comprises one or more gyroscopes 548. In embodiments, gyroscope 548 is configured to detect an orientation or change in orientation of CAD detection device 500. For example, if gyroscope 548 detects a change in orientation of CAD detection device 500 when CAD detection device 500 is in a data sense mode, it can be determined that CAD detection device 500 has tilted during sensing and the data measured during that point may have errors or otherwise be incorrect. Such orientation sensing can be utilized to properly place and position CAD detection device 500. In embodiments, an indication of a tilt can be provided to the user via the GUI of CAD detection device 500.

In an embodiment of CAD detection device 500 comprising gyroscope 548, noise determination can be conducted by determination of orientation or movement sensed by gyroscope 548. In embodiments, based on the amount of movement sensed by gyroscope 548, additional determination can be made of the source of the noise, such as lung noise, digestive noise, or movement of the device. In such embodiments, CAD detection device 500 can include only first acoustic sensor 526a, and need not include second acoustic sensor 526b for noise reduction or noise cancellation.

In an embodiment, CAD detection device 500 optionally further comprises an SD card port configured to receive an SD card or other portable memory. In such embodiments, CAD detection device 500 is effectively infinitely expandable such that an unlimited number of data sets can be stored. In embodiments, embedded non-volatile memory can be used in addition to or instead of those types of memory described herein.

In embodiments, CAD detection device 500 can be configured for gain control. In an embodiment, gain control can be automated. For example, an impedance measurement can detect abdominal loading or feedback and the gain adjusted appropriately. Gain can therefore be modified based on an impedance feedback. In embodiments, gain tuning or control can be based at least in part on data (e.g., patient weight, BMI, measurements, etc.) downloaded from an electronic health record (EHR). In embodiments, CAD detection device 500 can be pre-calibrated before use. Gain control can also based on real time acoustic measurement in embodiments, by analyzing a predetermined duration of acoustic signal (such as a few seconds) before any signal is stored into memory or analyzed.

Referring now to FIGS. 8A-C, a docking station 608 is shown according to one embodiment. Docking station 608, like docking station 108 previously described with respect to FIGS. 2A and 2B, can perform any of a variety of functions including but not limited to charging, protecting, calibrating, and/or diagnosing a CAD detection device. As shown in FIGS. 8A-C, docking station 608 includes a base 612, an acoustic pad or emitter 650, a set of pins 652, and an electromagnetic emitter 654.

Base 612 is similar to base 112 previously described with respect to FIGS. 2A and 2B, in that it is configured to interact with a CAD detection device. In embodiments, a CAD detection device can be positioned on base 612 whenever the device is not in use, or CAD detection device can be returned to base 612 as needed for charging, diagnostics, calibration, or data transfer purposes.

Base 612 is shaped to interface with a CAD detection device. For example, as previously depicted with respect to FIGS. 2A and 2B, a CAD detection device can have a substantially similar face such that base 612 and the CAD detection device can be positioned in intimate contact. In embodiments, securing mechanisms (such as retaining members 110a and 110b) can be used to prevent disconnection between a CAD detection device and base 612.

Base 612 can be connected to wired or wireless networks in embodiments, such that data at base 612 can be transmitted to a remote location. Base 612 can also include a cord and plug (not shown) in order to provide power for recharging a CAD detection device, in embodiments, or base 612 can contain a battery sufficient to recharge CAD detection device. In embodiments in which base 612 comprises a cord and plug, the plug can comprise a removable/interchangeable portion such that plug configurations used in different countries or regions can be swapped in and out of the plug easily and conveniently. In these embodiments, docking station 608 and device 102 can be compatible with and rated for voltages from 110V to 240V such that no voltage converter or transformer is necessary for operation anywhere in the world.

In alternative embodiments, such as is depicted in FIG. 8C, base 612 can include lights 620 or other indicators to show the status of a CAD detection device. In another example not depicted, base 612 itself can glow or light up. Various color schemes can be used, such as green when a corresponding CAD detection device 102 is charging or charged, red when it has failed a diagnostic exam, a flashing or other colored light if device 102 is not properly seated in docking station 608 or there is some other error or problem with charging, etc. In other embodiments, audible or other feedback can be provided instead of or in addition to lights 620. When CAD detection device 102 is removed from docking station 608, such as immediately after charging or between charging sessions, a start-up or other screen on display 616 can provide battery status or charging information in a variety of ways and formats. As depicted in FIG. 8D, display 116 shows that the battery has capacity to conduct ten patient scans. Instead of providing this information as patient scan number capacity, display 116 can display a percentage battery used or available, either as a number, a battery graphic or in some other way.

Acoustic pad or emitter 650, pins 652, and electromagnetic emitter 654 can be used in diagnostics, calibration, and/or data transfer to and from a CAD detection device, in embodiments. For example, CAD detection device 102, which is highly sensitive to noises and pressure changes, can be calibrated and checked for damage or changes in sensitivity or other characteristics over time by positioning the CAD detection device with its acoustic sensor (for example, acoustic sensor panel 126) adjacent to acoustic pad or emitter 650. Acoustic pad or emitter 650 can perform diagnostic checks of acoustic sensor panel 126 by emitting a test noise or noises, and verifying that CAD detection device measures the same signal that was output by acoustic pad or emitter 650. In some embodiments, acoustic pad or emitter 650 can comprise a sound tranducer.

In embodiments, acoustic pad or emitter 650 can also be used for calibration of acoustic sensors on the CAD detection device. In some embodiments, such as the embodiment previously described with respect to FIG. 3, a second acoustic sensor (121) is positioned opposite the main acoustic sensor (126). The combined output of these two sensors can be compared to the output of acoustic pad or emitter 650 to diagnose and/or calibrate the acoustic sensors 126 and 121. For example, in some embodiments the output of acoustic sensor 121 can be subtracted from the output of acoustic sensor 126, and compared to the signal provided by acoustic pad or emitter 650.

Referring to the embodiment of FIG. 8A, similar tests can be performed using pins 652 and electromagnetic emitters 654. Pins 652 can provide an electrical potential that is sensed by pin sensors 130a-130c of FIG. 5, for example. In addition, in some embodiments pins 652 can be used for data transfer to and from a CAD detection device, as well as charging of a CAD detection device. In the embodiment of FIG. 8B, docking station 608 comprises pogo pins 655a-655f to perform tests of ECG sensors 428a-428f (referring to the embodiment of FIG. 6C). An additional pogo pin 658 can be provided for grounding. Pin sensors 653 can interface with pin sensors 430 of device 102 (see FIG. 6C), as previously discussed.

In embodiments, docking station 608 can have various numbers of acoustic pads or emitters 650, sets of pins 652, and/or electromagnetic emitters 654. In the embodiment shown in FIG. 8A there are three sets of pins 652 and three electromagnetic emitters 654, and in FIG. 8B there is one set of six pins 652 and six pogo pins 655a-655f, but in alternative embodiments there could be, for example, six or twelve, or fewer or more, of either/each.

When testing or checking of device 102 by docking station 608, data can be collected and stored locally by docking station 608, though in other embodiments the data can be collected by or transmitted to, and stored by, device 102. The data then can be sent as a packet to a remote server or other location for analysis or review, and status or results information can be provided to a user via device 102 and/or docking station 608. For example, display 616 can display an icon (e.g., a check mark, plus sign, smiling emoticon or emoji, or other symbol or icon generally understood to indicate a positive result or status), text (e.g., “okay,” “OK,” “pass,” etc.), color (e.g., green), light or sound pattern, or other output when the condition or operation of device 102 is tested and confirmed to be in order. Display 616 can display a different icon (e.g., an “X,” a sad emoticon or emoji, or some other symbol or icon generally understood to indicate a negative results or status), text (“not okay,” “fail,” “contact customer service,” etc.), color (e.g., red), light or sound pattern, or other output when the condition or operation of device 102 is tested and confirmed to be in out of order or requiring attention. In other embodiments, some or all of the data analysis or diagnostics of device 102 can be carried out locally, without requiring data to be transmitted remotely.

Referring now to FIGS. 9A and 9B, a scan sequence guide 756 is depicted. CAD detection devices as previously described can be used in conjunction with an identification element, such scanning area identification pads or patient scan sequence guide 756, to aid in the proper placement of CAD detection device 400 while scanning a patient. Such systems are described in International Publication Nos. WO 2013023041, WO 2011071989, and U.S. Patent Pub. No. 2009/0177107, and U.S. Pat. No. 7,520,860, which have been incorporated herein by reference in their entireties.

In embodiments, sequence guide 756 can be coated in an anti-microbial coating for protection against disease. In embodiments, anti-microbial materials can be embedded within the layers of sequence guide 756.

In an embodiment, sequence guide 756 is initially folded or otherwise secured so that two or more sides can be secured with a seal 758, as depicted in FIG. 9B. In an embodiment, a broken seal indicates use of sequence guide 756. When seal 758 is broken, the set of a CAD detection device and/or a corresponding sequence guide 756 can be discarded as previously used. In another embodiment, sequence guide 756 comprises seal 758 including a wireless chip, such as an RFID chip, NFC chip, or other suitable hardware, to signal initiation to a CAD detection device. Activation of the chip of seal 758 can command a CAD detection device such as those previously described to begin initial processing prior to data collection.

In another embodiment, sequence guide 756 can be electromagnetically shielded such that data from wireless chips cannot be read from outside sequence guide 756. In an embodiment, the cover of sequence guide 756 is integrated with a shield. In an embodiment, the shield comprises conductive or magnetic materials, such as sheet metal, metal screen, and metal foam, or electromagnetically integrated paint or other coating.

In an embodiment of manufacturing sequence guide 756, RFID, NFC, or other wireless chips can be laid in a strip on the relevant scanning area identification pads. Such strip-based manufacturing saves time and money in producing sequence guide 756.

In an embodiment, sequence guide 756 can be integrated with a single removable patch. The patch can be removed from its coupling to sequence guide 756 and applied to a patient. In an embodiment, the patch comprises a sensor, circuitry, guide portion or other component at each relative location necessary to make a determination on the disease the system is intended to diagnose, such as coronary artery disease. For example, a single patch can be shaped to encompass all four locations (for example, those shown in FIG. 9A in the illustration of the patient chest) to determine coronary artery disease. A single “sample” event can then be conducted with all four locations once the patch is applied to the patient.

Sequence guide 756 can further comprise, in an embodiment, a display screen for displaying the results of the analysis or diagnosis of a particular disease. In other embodiments, a portion of sequence guide 756 comprises invisible ink that can likewise display the results of the analysis or diagnosis of a particular disease.

In still other embodiments, and referring to FIG. 9C, sequence guide 756 can be provided or presented electronically, such as via a tablet 770 or other device (e.g., a laptop, a computer, a smartphone, a smartwatch, a mobile communications device, or some other electronic or computing device). In these embodiments, sequence guide 756 can be animated and/or comprise both visual and audio information and prompts to a practitioner or other user of device 102. In some embodiments, tablet 770 can be coupled with device 102, wired or wirelessly, such that a user or practitioner can control operation of device 102 via tablet 770. For example, in one embodiment tablet and device 102 can be communicatively coupled with one another via BLUETOOTH, WiFi or some other wireless communications protocol. A CAD device application (“app”) or other software or program operating on tablet 770 can be used in conjunction with device 102 in embodiments. In some embodiments, the CAD app can be the only app operating on tablet 770, while in other embodiments the CAD app can “lock out” or prevent a user from accessing any other apps or features of tablet 770 during use. This feature can be controlled remotely (e.g., by a remote server affiliated with device 102) to prevent misuse or errors. The app generally will be a dedicated app and can facilitate automatic syncing and communications with device 102. To that end, a kit comprising tablet 770 preloaded with the app, at least one device 102, at least one docking station 608, and a user quick start guide can be assembled, packaged and provided in embodiments. In still other embodiments, the app may be downloadable via an online app store or other source. A code or access information may be provided with device 102 to enable downloading of and/or access to the app. A subscription may be required to access sequence guides and other features of the app.

Sequence guide 756, via the app and tablet 770, can present user input/output (I/O) features, such as user authentication I/Os (e.g., login/password, biometrics such as fingerprint or retinal scanning, or facial or voice recognition), as well as buttons or other features that look similar to inputs 118a-118c of device 102 for control of device 102 during use. In some embodiments, tablet 770 can display an image of a device 102 such that a user can press buttons on the image (via a touchscreen of tablet 770) with the same effect of having pressed the same buttons physically located on device 102. In other embodiments, an interactive version of sequence guide 756 is displayed, such that a user can touch “START” on sequence guide 756 via a touchscreen of tablet 770, and the command wirelessly communicated to device 102 such that device 102 anticipates a patient scanning sequence to begin or automatically begins collecting data. In still other embodiments, voice recognition of user commands via tablet 770 can be implemented.

In some embodiments, tablet 770 can provide user calibration features. For example, tablet 770 can comprise a gyroscope and/or accelerometer to calibrate hand movement or vibration of a user via tablet 770 in order to improve data collection via device 102. In other embodiments, these features (e.g., a gyroscope and/or accelerometer) can be used for user training so that users can receive feedback regarding positioning, pressure, movement and use of device 102 before using it with a patient. This feedback can be haptic, visual and/or audible and include an auto-prompt for user adjustment related to one or more of user grip, device pressure on the patient, tilt, etc. In these cases, tablet 770 can comprise one or more separate apps for training, calibration, and other features and functions.

During patient scans or other operation, tablet 770 can provide audible and/or visual prompts to a user. For example, tablet 770 can prompt a user to move to a next scanning area on a patient when sufficient data at a current location is obtained, or to rescan or restart scanning at a location if insufficient or bad (e.g., noisy or low quality) data is being collected. In such a situation, tablet 770 and/or device 102 can stop collecting data so that memory, battery and other resources are not used to collect, store or transmit bad data. As previously discussed, device 102 can comprise microphone 121, though in other embodiments tablet 770 can, additionally or alternatively, also comprise or couple with (e.g., via an external jack or port) a microphone or other sensor to detect or measure ambient noise that could affect operation of device 102. Tablet 770 can be configured to cause device 102 to stop collecting data in noisy situations and/or to provide feedback to a user (e.g., via a noise meter displayed on tablet 770) so that noise can be reduced and data collection restart or continue.

In still other embodiments, tablet 770 can comprise or be coupled with other sensors or devices to improve or enhance operation of device 102. For example, in an embodiment one or more ECG sensors can be incorporated in or coupled with tablet 770 to be used in cooperation with ECG or other sensors of device 102. Six ECG sensors can be located on device 102, and six ECG sensors can be located on or coupled with tablet 770 to achieve a twelve-sensor ECG array. These and other sensors (e.g., microphone, temperature, etc.) can be incorporated directly in tablet 770 or coupled with tablet 770, wired (e.g., via an external port or jack of tablet 770) or wirelessly (e.g., via BLUETOOTH, WiFi, or another wireless communications protocol).

In another embodiment, and referring to FIG. 9D, data collection device 102 can be operated in conjuction with a virtual or augmented reality system, such as one comprising augmented relatity glasses, goggles or other hardware. In one embodiment, an app running on tablet 770 can facilitate or complement use of the augmented reality system with device 102, though in other embodiments the augmented reality system can operate independently with device 102. During operation of device 102, the augmented reality system can display data or other information, such as data or information otherwise viewable on tablet 770, in a viewscreen or viewing area of a user wearing the glasses or goggles. An example of a user's augmented view when using an augmented reality system with device 102 is depicted in FIG. 9D. Here, the augmented reality system can present information 790 in the user's view to guide the user to place or position device 102 on the correct patient thorax location and can also provide realtime data quality feedback and or instantaneous diagnoses and other information. This can assist a user in use and operation of device 102, as the user need not look down or turn to view tablet 770 when collecting data from a patient. Prompts, instructions, sequence guide 756, and other information also can be displayed via the augmented reality system.

Tablet 770 also can function as an extension of display 616 of device 102. For example, display 616 can display an icon or prompt, and tablet 770 can display additional and more detailed information. This feature can provide advantages to users without sacrificing battery or other limited resources of device 102.

Once sufficient patient data is collected, data can be sent from tablet 776 (or, additionally or alternatively, from device 102 or docking station 108) to networked or local computer system 106. In some embodiments, this can be accomplished via data portal 104 (see FIG. 1). The data then can be processed, as discussed below, to produce a report. In some embodiments, the processing is conducted via networked or local computer system 106. In other embodiments, staged processing is used, with some information processed by networked or local computer system 106, and some via one or both of device 102 and tablet 770. Encryption and decryption techniques can be used at various stages, such as using encryption when transferring data between device 102 and tablet 770, between device 102 and networked or local computer system 106, between tablet 770 and networked or local computer system 106, and in other situations in which external sensors or other devices are used (e.g., such as when coupled with tablet 770).

When a report is complete, however it is processed and obtained, the report can be sent back to and presented via tablet 770. Here, too, encryption and decryption techniques can be used when transferring the report and related data and information. In some embodiments, the report can be automatically displayed on tablet 770 when it is available. In other embodiments, tablet 770 can access and display a remote report, without the report having to be sent to tablet 770. In still other embodiments, the report can, additionally or alternatively, be sent (e.g., via email) to another address or location remote from tablet 770. In a further embodiment, the report or other results or information related to a patient can be sent directly to or incorporated in a patient electronic medical record (EMR).

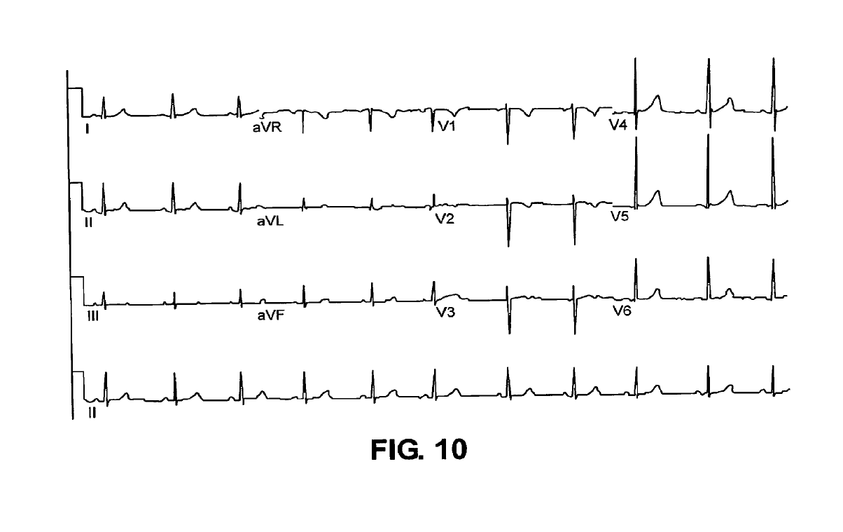

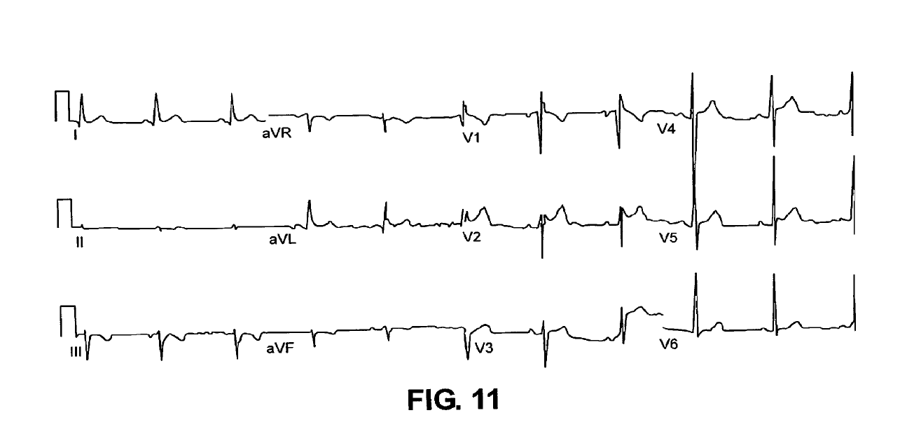

FIGS. 10 and 11 depict ECG data acquired in the four positions of a patient as indicated in sequence guide 756 of FIGS. 9A-9C. FIGS. 10 and 11 each show ECG data relating to the Subxyphoid, 4LICS Parasternal border, 2LICS Parasternal Border, and 2LICS Mid Clavicular regions of a human heart. The scans depicted in FIG. 10 correspond to a healthy human heart, whereas the scans depicted in FIG. 11 correspond to a diseased human heart.

ECG scan data, such as the scan data depicted in FIGS. 10 and 11, can be used to identify abnormalities in heart function, for example. Some scans can provide sufficient data to identify or diagnose a particular heart condition. While four scan positions are shown in FIGS. 10 and 11, in alternative embodiments more or fewer scanned areas can be analyzed together. Likewise, while each of the four scan positions shown in FIGS. 10 and 11 are scanned three times, in alternative embodiments where more precision is desired a test could include more or longer scans, whereas a faster test may have fewer or shorter scans.

The ECG scan data shown in FIGS. 10 and 11 can be combined or considered with acoustic data, in embodiments. For example, acoustic data collected by acoustic sensor panel 126 (FIG. 3) can be combined with ECG scan data to increase the accuracy or predictive power of ECG scan data. For example, in embodiments an arrhythmia or turbulence detected by an acoustic sensor can correspond with electrical abnormalities in the ECG scan data, indicating a particular heart disorder or blockage.

In embodiments, ECG sensor data and acoustic sensor data can be combined or co-considered to provide an indication of a particular disorder, or of a likelihood of a particular disorder, or simply that the results are abnormal. In some cases, the data can indicate that subsequent angiography or even surgical intervention is recommended.

In embodiments, networked or local computer system 106 (FIGS. 1, 9C) can store raw and/or anonymized data relating to ECG scans and acoustic scans. Such data can be stored both independently (e.g., a database of raw ECG scan data can be stored on networked or local computer system 106) or in a combined format (e.g., a database of ECG scan data and contemporaneously-acquired acoustic scan data can be stored on networked or local computer system 106). These data can be assembled and stored as combined ECG/acoustic data in raw and/or processed forms. In embodiments, these data, as well as the results of subsequent angiography or other information, can be used to enhance the predictive power of the ECG scans and acoustic data. For example, a particular feature or interaction of ECG and acoustic data at networked or local computer system 106 can correspond to a high likelihood of a particular type of blockage or defect. With increasing amounts of scan data, the predictive power and statistical significance of the ECG and acoustic data also increases. This predictive power can be used in machine learning implementations, such as in data collection or processing, to improve results in future cases.

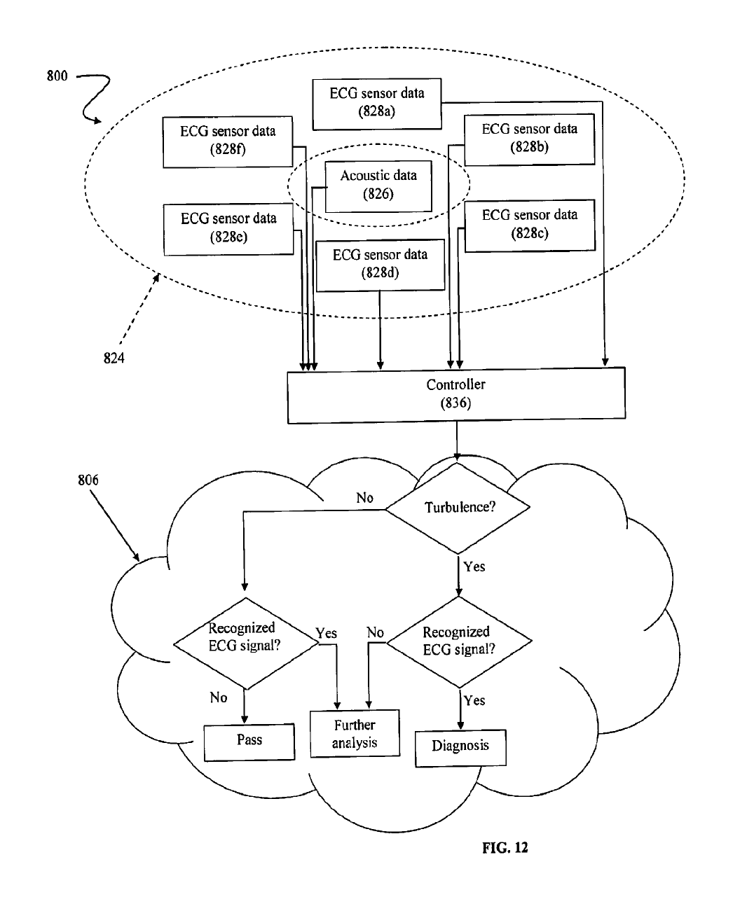

FIG. 12 is a flowchart of a system 800 for diagnosing heart health, according to an embodiment. In the embodiment shown in FIG. 12, the diagnosis can utilize both acoustic data (e.g., turbulence) and ECG data together to make a diagnosis about presence of turbulence as well as presence of ECG abnormality. In embodiments, the presence of turbulence detected by the acoustic sensor(s) and the location of an ECG-detected abnormality can be used in combination to provide a diagnosis. In another embodiment, the turbulence lends a diagnosis separate from the ECG diagnosis, as both can provide individual information. Together they can provide more specificity than either type of test could provide in isolation, in embodiments.

As shown in FIG. 12, acoustic data 826 (acquired from an acoustic panel such as acoustic sensor panel 126) and ECG sensor data 826a-826f (acquired from ECG sensor panel 824) are routed to controller 836. Controller 836 can send these data for remote processing at networked or local computer system 806, in embodiments. Local processing, in whole or in part, can also be done in embodiments. At networked or local computer system 806, if turbulence is detected in the acoustic data 826 and an abnormal ECG signal is recognized, a diagnosis can be provided by system 800. Alternatively, where no turbulence is detected but an ECG signal is recognized, or where turbulence is detected but no ECG signal is recognized, further analysis can be recommended. Further analysis could include, for example, angiogram. In scenarios where ECG signal does not correspond to a recognized defect pattern and where turbulence is not detected, no further test is recommended.

The systems described herein, including system 800, provide contemporaneously-acquired electrical and acoustic data. This contemporaneous data in two modes can diagnose cardiac and/or pulmonary conditions quickly and accurately, without the need for invasive and expensive alternatives such as angiography. Embodiments are small, portable, and versatile, and can be used for both human medical evaluations and in veterinary applications.

As used herein, the term “processor” can refer to any suitable programmable device that accepts digital data as input, is configured to process the input according to instructions or algorithms, and provides results as outputs. In an embodiment, the processor can be a central processing unit (CPU) configured to carry out the instructions of a computer program. In other embodiments, the processor can be an Advanced RISC (Reduced Instruction Set Computing) Machine (ARM) processor or other embedded microprocessor. In other embodiments, the processor comprises a multi-processor cluster. The processor is therefore configured to perform at least basic selected arithmetical, logical, and input/output operations.

Memory can comprise volatile or non-volatile memory as required by the coupled processor to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing examples in no way limit the type of memory that can be used, as these embodiments are given only by way of example and are not intended to limit subject matter hereof In other embodiments, the memory comprises a plurality of memory.

Various embodiments of systems, devices, and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A data collection device for generating an indication of health, comprising:
a first acoustic sensor configured to generate a first acoustic data output;
at least one electrocardiogram (ECG) sensor, wherein the at least one ECG sensor is configured to generate an ECG sensor output; and
a processor configured to concurrently analyze the ECG sensor output and the acoustic sensor output to obtain and provide a qualitative result related to an indication of an anatomical or physiological characteristic.

2. The data collection device of claim 1, further comprising communications circuitry coupled to the controller, wherein the controller is configured to transmit the acoustic sensor output and the ECG sensor output to a remote processor.

3. (canceled)

4. The data collection device of claim 1, wherein each of the at least one ECG sensors is arranged on a common face of the data collection device.

5. The data collection device of claim 4, wherein the acoustic sensor is arranged on the common face of the device.

6. The data collection device of claim 1, further comprising at least three ECG sensors.

7. (canceled)

8. The data collection device of claim 4, further comprising a second acoustic sensor configured to generate a second acoustic data output, wherein the second acoustic sensor is arranged on a surface of the data collection device opposite the common face.

9. (canceled)

10. The data collection device of claim 8, wherein the controller is configured to combine the first acoustic sensor output and the second acoustic sensor output to correct for noises.

11. (canceled)

12. The data collection device of claim 1, further comprising a plurality of ground contacts, wherein the ground contacts are interdigitated with the at least one ECG sensor.

13. (canceled)

14. The data collection device of claim 1, further comprising a ground plane shared by the at least one ECG sensors.

15. The data collection device of claim 1, further comprising a plurality of pin sensors configured to do at least one of: receive electrical power to charge a battery of the system, and send or receive data from an external device.

16. (canceled)

17. A system for analyzing heart health, the system comprising:
a data collection device having a first acoustic sensor configured to generate a first acoustic data output;
at least one electrocardiogram (ECG) sensor configured to generate an ECG sensor output;
a controller configured to combine the ECG sensor output and the acoustic sensor output to provide an indication of an anatomical or physiological condition; and
a docking station configured to mechanically and electrically interface with the acoustic sensor and the at least one ECG sensor, wherein the docking station comprises:
a base;
an acoustic emitter arranged on the base;
pins arranged on the base; and
an ECG sensor interface.

18. (canceled)

19. The system of claim 17, wherein the data collection device is configured to mechanically interface with the base such that:
the acoustic emitter is arranged adjacent to the first acoustic sensor;
the pins are arranged adjacent to a plurality of pin receivers of the data collection device; and
the electromagnetic emitter is arranged adjacent to a second portion of the plurality of ECG sensors.

20. The system of claim 17, wherein the at least one ECG sensor comprises a plurality of ECG sensors arranged on a common surface of the data collection device.

21. The system of claim 17, further comprising a portable computing device communicatively coupleable with at least one of the data collection device or the docking station, wherein a first plurality of the at least one ECG sensor is arranged on a common surface of the data collection device, and a second plurality of the at least one ECG sensor is arranged at the portable computing device.

22. (canceled)

23. (canceled)

24. The system of claim 21, wherein the portable computing device comprises a microphone to sense ambient noise proximate the data collection device.

25. The system of claim 21, further comprising a remote processing device communicatively coupled with at least one of the portable computing device, the data collection device, or the docking station, wherein the remote processing device and at least one of the portable computing device, the data collection device, or the docking station are configured for staged processing of the first acoustic data output and the ECG sensor output.

26. (canceled)

27. A method for detecting a health condition with a data collection device, the method comprising:
providing a device including a first acoustic sensor configured to generate a first acoustic data output, at least one electrocardiogram (ECG) sensor, wherein the at least one ECG sensor is configured to generate an ECG data output, and a processor configured to concurrently collect the ECG data output and the acoustic data output;
providing a networked computer system configured to receive the ECG data output and the acoustic data output from the device, wherein the networked computer system is further configured to provide an indication of an anatomical or physiological characteristic by determining whether at least one of the following is present:
a known ECG pattern corresponding to the health condition is present in the ECG data output and determining whether a known acoustic pattern corresponding to the health condition is present in the acoustic data output, or
at least one known pattern based on both the ECG data output and the acoustic data output is present;
providing an output related to the determining; and
providing a base configured to calibrate the device by:
arranging the ECG sensors and the acoustic sensor on a base;
providing an acoustic test signal from the base to the acoustic sensor;
providing a test signal from the base to the ECG sensors;
comparing the provided test signals to those sensed by the acoustic sensor and ECG sensors; and
calibrating the acoustic sensor and the ECG sensors based on the comparison.

28. The method of claim 27, wherein providing the output comprises:
providing a positive indication that the health condition is likely if the acoustic data output and the ECG data output both correspond to the health condition;
providing a negative indication that the health condition is likely if the acoustic data output and the ECG data output both do not correspond to the health condition; and
providing a recommendation for further testing if either the ECG data output or the acoustic data output are inconclusive as to the health condition.

29. (canceled)

30. The method of claim 27, further comprising disabling the data collection device if the comparison shows a malfunctioning sensor.

31. (canceled)

32. The method of claim 27, further comprising:
receiving, by the networked computer system, data related to movement of a user of the data collection device; and
considering the data related to movement of the user in the determining.

33. (canceled)
